(19) United States
(12) Patent Application Publication
(10) Pub. No.: US 2012/0029313 A1
(43) Pub. Date: Feb. 2, 2012

(54) SYSTEM AND METHOD FOR TRACKING VITAL-SIGNS MONITOR PATCHES

(76) Inventors: Alison Burdett, Oxford (GB); Damitha Wilwara Arachchige, Abingdon (GB); Mat Key, Oxford (GB)

(21) Appl. No.: 12/844,788

(22) Filed: Jul. 27, 2010

(51) Int. Cl. A61B 5/00

(52) U.S. Cl. 600/301

(57) ABSTRACT

Systems and methods of tracking vital-sign monitor patches are disclosed. At least one of a new wireless communication link and loss of an existing wireless communication link between a vital-sign monitor patch and a bridge in the monitoring network is identified. A database comprising information indicative of wireless communication links between vital-sign monitor patches and bridges in the monitoring network is accessed. The information in the database is updated to indicate the new wireless communication link or the loss of the existing wireless communication link.

### CROSS-REFERENCES TO RELATED APPLICATIONS

The following applications disclose certain common subject matter with the present application: A Vital-Signs Monitor with Encapsulation Arrangement, docket number 080624-0612; A Vital-Signs Monitor with Spaced Electrodes, docket number 080624-0623; A Vital-Signs Patch Having a Strain Relief, docket number 080624-0624; A Temperature Probe Suitable for Axillary Reading, docket number 080624-0625; System and Method for Monitoring Body Temperature of a Person, docket number 080624-0626; A System and Method for Storing and Forwarding Data from a Vital-Signs Monitor, docket number 080624-0627; System and Method for Saving Battery Power in a Vital Signs Monitor, docket number 080624-0628; A System and Method for Conserving Battery Power in a Patient Monitoring System, docket number 080624-0629; A System and Method for Saving Battery Power in a Patient Monitoring System, docket number 080624-0630; A System And Method for Reducing False Alarms Associated with Vital-Signs Monitoring, docket number 080624-0632; A System And Method for Location Tracking of Patients in a Vital-Signs Monitoring System, docket number 080624-0633; A System And Method for Reducing False Alarms Based on Motion and Location Sensing, docket number 080624-0634; all of the listed applications filed on ______.

### FIELD

The present disclosure generally relates to systems and methods of physiological monitoring, and, in particular, a system and method for tracking vital-signs monitor patches.

### DESCRIPTION OF THE RELATED ART

Some of the most basic indicators of a person's health are those physiological measurements that reflect basic body functions and are commonly referred to as a person's “vital signs.” The four measurements commonly considered to be vital signs are body temperature, pulse rate, blood pressure, and respiratory rate. Some clinicians consider oxygen saturation (S₀₂) to be a “fifth vital sign” particularly for pediatric or geriatric cases. Some or all of these measurements may be performed routinely upon a patient when they arrive at a healthcare facility, whether it is a routine visit to their doctor or arrival at an Emergency Room (ER).

Vital signs are frequently taken by a nurse using basic tools including a thermometer to measure body temperature, a sphygmomanometer to measure blood pressure, and a watch to count the number of breaths or the number of heart beats in a defined period of time which is then converted to a “per minute” rate. If a patient's pulse is weak, it may not be possible to detect a pulse by hand and the nurse may use a stethoscope to amplify the sound of the patient's heart beat so that she can count the beats. Oxygen saturation of the blood is most easily measured with a pulse oximeter.

When a patient is admitted to a hospital, it is common for vital signs to be measured and recorded at regular intervals during the patient's stay to monitor their condition. A typical interval is 4 hours, which leads to the undesirable requirement for a nurse to awaken a patient in the middle of the night to take vital sign measurements.

When a patient is admitted to an ER, it is common for a nurse to do a “triage” assessment of the patient's condition that will determine how quickly the patient receives treatment. During busy times in an ER, a patient who does not appear to have a life-threatening injury may wait for hours until more-serious cases have been treated. While the patient may be reassessed at intervals while awaiting treatment, the patient may not be under observation between these reassessments.

Measuring certain vital signs is normally intrusive at best and difficult to do on a continuous basis. Measurement of body temperature, for example, is commonly done by placing an oral thermometer under the tongue or placing an infrared thermometer in the ear canal such that the tympanic membrane, which shared blood circulation with the brain, is in the sensor's field of view. Another method of taking a body temperature is by placing a thermometer under the arm, referred to as an “axillary” measurement as axilla is the Latin word for armpit. Skin temperature can be measured using a stick-on strip that may contain panels that change color to indicate the temperature of the skin below the strip.

Measurement of respiration is easy for a nurse to do, but relatively complicated for equipment to achieve. A method of automatically measuring respiration is to encircle the upper torso with a flexible band that can detect the physical expansion of the rib cage when a patient inhales. An alternate technique is to measure a high-frequency electrical impedance between two electrodes placed on the torso and detect the change in impedance created when the lungs fill with air. The electrodes are typically placed on opposite sides of one or both lungs, resulting in placement on the front and back or on the left and right sides of the torso, commonly done with adhesive electrodes connected by wires or by using a torso band with multiple electrodes in the strap.

Measurement of pulse is also relatively easy for a nurse to do and intrusive for equipment to achieve. A common automatic method of measuring a pulse is to use an electrocardiograph (ECG or EKG) to detect the electrical activity of the heart. An EKG machine may use 12 electrodes placed at defined points on the body to detect various signals associated with the heart function. Another common piece of equipment is simply called a “heart rate monitor.” Widely sold for use in exercise and training, heart rate monitors commonly consist of a torso band, in which are embedded two electrodes held against the skin and a small electronics package. Such heart rate monitors can communicate wirelessly to other equipment such as a small device that is worn like a wristwatch and that can transfer data wirelessly to a PC.

Nurses are expected to provide complete care to an assigned number of patients. The workload of a typical nurse is increasing, driven by a combination of a continuing shortage of nurses, an increase in the number of formal procedures that must be followed, and an expectation of increased documentation. Replacing the manual measurement and logging of vital signs with a system that measures and records vital signs would enable a nurse to spend more time on other activities and avoid the potential for error that is inherent in any manual procedure.

### SUMMARY

For some or all of the reasons listed above, there is a need to be able to continuously monitor patients in different settings. In addition, it is desirable for this monitoring to be done with limited interference with a patient's mobility or interfering with their other activities.

Embodiments of the patient monitoring system disclosed herein measure certain vital signs of a patient, which include respiratory rate, pulse rate, blood pressure, body temperature, and, in some cases, oxygen saturation (S_{O2}), on a regular basis and compare these measurements to defined limits.

In one aspect of the present disclosure, a method of tracking vital-sign monitor patches in a vital-sign monitoring network is provided. The method can comprise identifying at least one of a new wireless communication link and loss of an existing wireless communication link between a vital-sign monitor patch and a bridge in the monitoring network. The method can further comprise accessing a database comprising information indicative of wireless communication links between vital-sign monitor patches and bridges in the monitoring network. The method can further comprise updating the information in the database to indicate the new wireless communication link or the loss of the existing wireless communication link.

In one aspect of the present disclosure, a vital-sign monitoring system is provided. The system can comprise a plurality of vital-sign monitor patches configured to monitor one or more vital signs of patients to whom the vital-sign monitor patches are attached. The system can further comprise a surveillance server configured to gather data relating to the one or more vital signs of the patients from the plurality of vital-sign monitor patches. The system can further comprise a plurality of bridges configured to provide data connections between the plurality of vital-sign monitor patches and the surveillance server. The system can further comprise a database configured to store information indicative of wireless communication links between at least some of the plurality of vital-sign monitor patches and at least some of the plurality of bridges. The database can be updated to indicate a new wireless communication link or loss of an existing wireless communication link between a vital-sign monitor patch and a bridge.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 is a diagram illustrating an exemplary embodiment of a patient monitoring system according to certain aspects of the present disclosure.

FIG. 2A is a perspective view of the vital-signs monitor patch of FIG. 1 according to certain aspects of the present disclosure.

FIG. 2B is a cross-section of the vital-signs monitor patch of FIG. 1 according to certain aspects of the present disclosure.

FIG. 2C is a functional block diagram illustrating exemplary electronic and sensor components of the vital-signs monitor patch of FIG. 1 according to certain aspects of the present disclosure.

FIG. 3A is a functional schematic diagram of the bridge according to certain aspects of the subject disclosure.

FIG. 3B is a functional schematic diagram of an embodiment of the surveillance server according to certain aspects of the present disclosure.

FIG. 4A is a map depicting a healthcare facility (e.g., a hospital) 400 in which a patient monitoring system such as the one shown in FIG. 1 is implemented according to certain aspects of the present disclosure.

FIG. 4B is a portion of an exemplary database comprising monitor patches, their linkable bridges, signal levels associated with the communication links between the monitor patches and the linkable bridges, and selected bridges according to certain aspects of the present disclosure.

FIG. 5A is a map of the healthcare facility depicted in FIG. 4A after a passage of time.

FIG. 5B is a portion of an updated version of the database shown in FIG. 4B according to certain embodiments of the present disclosure.

FIGS. 6A-C show a first set of lists stored in various bridge at the time of FIG. 4A, and a second set of lists which corresponds to updated lists stored in the bridges at the time of FIG. 5A.

FIG. 7 is a flowchart illustrating a process for tracking locations of monitor patches by keeping and updating a database comprising information indicative of the monitor patches and their linkable and selected bridges according to certain aspects of the present disclosure.

FIG. 8A is a diagram illustrating an exemplary data structure for a message indicating a new communication link and/or loss of an existing communication link according to certain aspects of the present disclosure.

FIG. 8B is a diagram illustrating an exemplary data structure for an alternative message indicating a new communication link and/or loss of an existing communication link according to alternative aspects of the present disclosure.

FIG. 9 is a flowchart illustrating an exemplary process for a bridge selection process according to certain aspects of the present disclosure.

FIG. 10 is a flowchart illustrating a process for detecting an inoperable bridge and selecting an alternative bridge to replace the inoperable bridge for the monitor patches that were previously associated with the inoperable bridge according to certain aspects of the present disclosure.

FIG. 11 is a flowchart illustrating a process for determining locations of patients in a healthcare facility according to certain aspects of the present disclosure.

FIG. 12A is an exemplary database comprising monitor patches, IDs of patients assigned to the monitor patches, and names of assigned patients according to certain aspects of the present disclosure.

FIG. 12B is an exemplary database comprising bridges and their respective locations within the healthcare facility according to certain aspects of the present disclosure.

### DETAILED DESCRIPTION

Periodic monitoring of patients in a hospital is desirable at least to ensure that patients do not suffer an un-noticed sudden deterioration in their condition or a secondary injury during their stay in the hospital. It is impractical to provide continuous monitoring by a clinician and cumbersome to connect sensors to a patient, which are then connected to a fixed monitoring instrument by wires. Furthermore, systems that sound an alarm when the measured value exceeds a threshold value may sound alarms so often and in situations that are not truly serious that such alarms are ignored by clinicians.

Measuring vital signs is difficult to do on a continuous basis. Accurate measurement of cardiac pulse, for example, can be done using an electrocardiograph (ECG or EKG) to detect the electrical activity of the heart. An EKG machine may use up to 12 electrodes placed at various points on the body to detect various signals associated with the cardiac function. Another common piece of equipment is termed a “heart rate monitor.” Widely sold for use in exercise and physical training, heart rate monitors may comprise a torso band in which are embedded two electrodes held against the skin and a small electronics package. Such heart rate monitors can communicate wirelessly to other equipment such as a small device that is worn like a wristwatch and that can transfer data wirelessly to a personal computer (PC).

Monitoring of patients that is referred to as “continuous” is frequently periodic, in that measurements are taken at intervals. In many cases, the process to make a single measurement takes a certain amount of time, such that even back-to-back measurements produce values at an interval equal to the time that it takes to make the measurement. For the purpose of vital sign measurement, a sequence of repeated measurements can be considered to be “continuous” when the vital sign is not likely to change an amount that is of clinical significance within the interval between measurements. For example, a measurement of blood pressure every 10 minutes may be considered “continuous” if it is considered unlikely that a patient's blood pressure can change by a clinically significant amount within 10 minutes. The interval appropriate for measurements to be considered continuous may depend on a variety of factors including the type of injury or treatment and the patient's medical history. Compared to intervals of 4-8 hours for manual vital sign measurement in a hospital, measurement intervals of 30 minutes to several hours may still be considered “continuous.”

Certain exemplary embodiments of the present disclosure include a system that comprises a vital-signs monitor patch that is attached to the patient, and a bridge that communicates with monitor patches and links them to a central server that processes the data, where the server can send data and alarms to a hospital system according to algorithms and protocols defined by the hospital.

The construction of the vital-signs monitor patch is described according to certain aspects of the present disclosure. As the patch may be worn continuously for a period of time that may be several days, as is described in the following disclosure, it is desirable to encapsulate the components of the patch such that the patient can bathe or shower and engage in their normal activities without degradation of the patch function. An exemplary configuration of the construction of the patch to provide a hermetically sealed enclosure about the electronics is disclosed.

In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

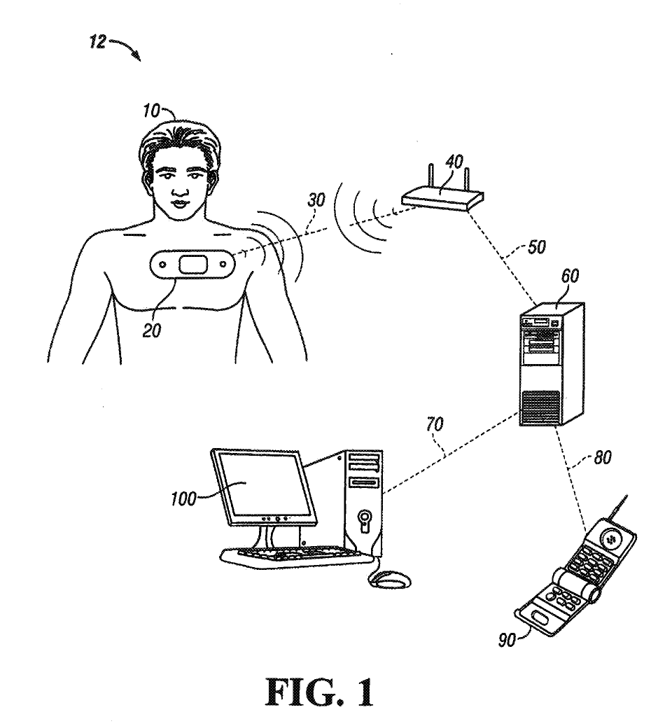

FIG. 1 discloses a vital sign monitoring system according to certain embodiments of the present disclosure. The vital sign monitoring system 12 includes vital-signs monitor patch 20, bridge 40, and surveillance server 60 that can send messages or interact with peripheral devices exemplified by mobile device 90 and workstation 100.

Monitor patch 20 resembles a large adhesive bandage and is applied to a patient 10 when in use. It is preferable to apply the monitor patch 20 to the upper chest of the patient 10 although other locations may be appropriate in some circumstances. Monitor patch 20 incorporates one or more electrodes (not shown) that are in contact with the skin of patient 10 to measure vital signs such as cardiac pulse rate and respiration rate. Monitor patch 20 also may include other sensors such as an accelerometer, temperature sensor, or oxygen saturation sensor to measure other characteristics associated with the patient. These other sensors may be internal to the monitor patch 20 or external sensors that are operably connected to the monitor patch 20 via a cable or wireless connection. Monitor patch 20 also includes a wireless transmitter that can both transmit and receive signals. This transmitter is preferably a short-range, low-power radio frequency (RF) device operating in one of the unlicensed radio bands. One band in the United States (US) is, for example, centered at 915 MHz and designated for industrial, scientific and medical (ISM) purposes. An example of an equivalent band in the European Union (EU) is centered at 868 MHz. Other frequencies of operation may be possible dependent upon the International Telecommunication Union (ITU), local regulations and interference from other wireless devices.

Surveillance server 60 may be a standard or virtualized computer server connected to the hospital communication network and preferably located in the hospital data center or computer room, although other locations may be employed. The server 60 stores and processes signals related to the operation of the patient monitoring system 12 disclosed herein including the association of individual monitor patches 20 with patients 10 and measurement signals received from multiple monitor patches 20. Hence, although only a single patient 10 and monitor patch 20 are depicted in FIG. 1, the server 60 is able to monitor the monitor patches 20 for multiple patients 10.

Bridge 40 is a device that connects, or “bridges”, between monitor patch 20 and server 60. Bridge 40 communicates with monitor patch 20 over communication link 30 operating, in these exemplary embodiments, at approximately 915 MHz and at a power level that enables communication link 30 to function up to a distance of approximately 10 meters. It is preferable to place a bridge 40 in each room and at regular intervals along hallways of the healthcare facility where it is desired to provide the ability to communicate with monitor patches 20. Bridge 40 also is able to communicate with server 60 over network link 50 using any of a variety of computer communication systems including hardwired and wireless Ethernet using protocols such as 802.11a/b/g or 802.3af. As the communication protocols of communication link 30 and network link 50 may be very different, bridge 40 provides data buffering and protocol conversion to enable bidirectional signal transmission between monitor patch 20 and server 60.

While the embodiments illustrated by FIG. 1 employ a bridge 20 to provide communication link between the monitor patch 20 and the server 60, in certain alternative embodiments, the monitor patch 20 may engage in direct wireless communication with the server 60. In such alternative embodiments, the server 60 itself or a wireless modem connected to the server 60 may include a wireless communication system to receive data from the monitor patch 20.

In use, a monitor patch 20 is applied to a patient 10 by a clinician when it is desirable to continuously monitor basic vital signs of patient 10 while patient 10 is, in this embodiment, in a hospital. Monitor patch 20 is intended to remain attached to patient 10 for an extended period of time, for example, up to 5 days in certain embodiments, limited by the battery life of monitor patch 20. In some embodiments, monitor patch 20 is disposable when removed from patient 10.

Server 60 executes analytical protocols on the measurement data that it receives from monitor patch 20 and provides this information to clinicians through external workstations 100, preferably personal computers (PCs), laptops, or smart phones, over the hospital network 70. Server 60 may also send messages to mobile devices 90, such as cell phones or pagers, over a mobile device link 80 if a measurement signal exceeds specified parameters. Mobile device link 80 may include the hospital network 70 and internal or external wireless communication systems that are capable of sending messages that can be received by mobile devices 90.

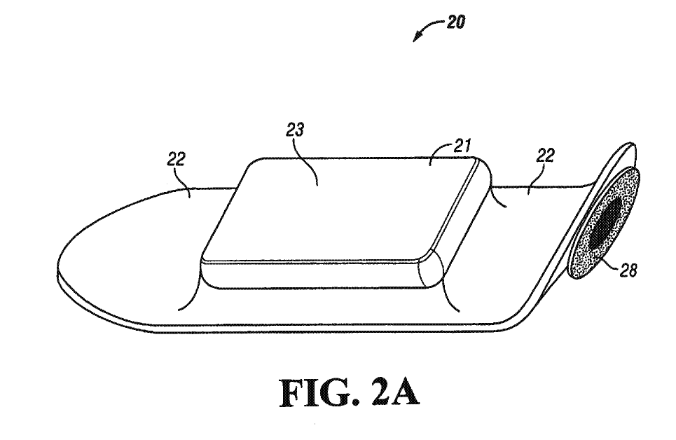

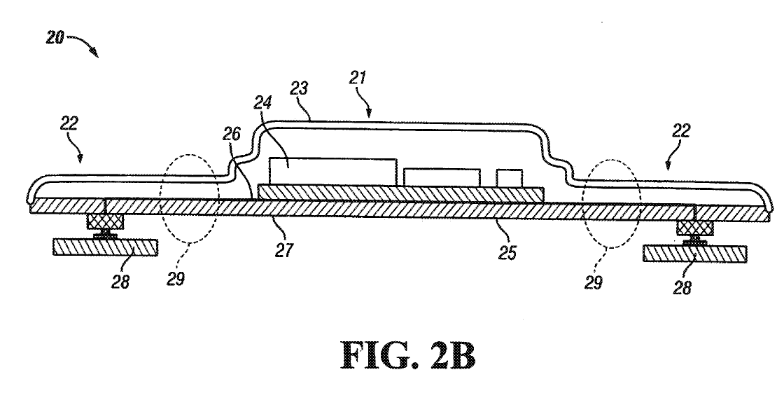

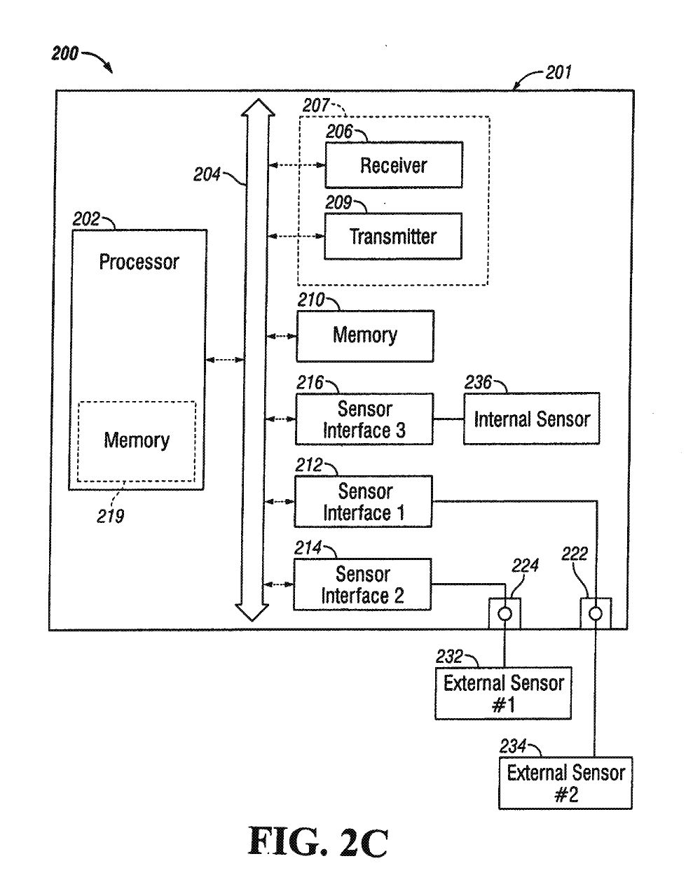

FIG. 2A is a perspective view of the vital-signs monitor patch 20 shown in FIG. 1 according to certain aspects of the present disclosure. In the illustrated embodiment, the monitor patch 20 includes component carrier 23 comprising a central segment 21 and side segments 22 on opposing sides of the central segment 21. In certain embodiments, the central segment 21 is substantially rigid and includes a circuit assembly (24, FIG. 2B) having electronic components and battery mounted to a rigid printed circuit board (PCB). The side segments 22 are flexible and include a flexible conductive circuit (26, FIG. 2B) that connect the circuit assembly 24 to electrodes 28 disposed at each end of the monitor patch 20, with side segment 22 on the right shown as being bent upwards for purposes of illustration to make one of the electrodes 28 visible in this view.

FIG. 2B is a cross-sectional view of the vital-signs patch 20 shown in FIGS. 1 and 2A according to certain aspects of the present disclosure. The circuit assembly 24 and flexible conductive circuit 26 described above can be seen herein. The flexible conductive circuit 26 operably connects the circuit assembly 24 to the electrodes 28. Top and bottom layers 23 and 27 form a housing 25 that encapsulate circuit assembly 28 to provide a water and particulate barrier as well as mechanical protection. There are sealing areas on layers 23 and 27 that encircles circuit assembly 28 and is visible in the cross-section view of FIG. 2B as areas 29. Layers 23 and 27 are sealed to each other in this area to form a substantially hermetic seal. Within the context of certain aspects of the present disclosure, the term ‘hermetic’ implies that the rate of transmission of moisture through the seal is substantially the same as through the material of the layers that are sealed to each other, and further implies that the size of particulates that can pass through the seal are below the size that can have a significant effect on circuit assembly 24. Flexible conductive circuit 26 passes through portions of sealing areas 29 and the seal between layers 23 and 27 is maintained by sealing of layers 23 and 27 to flexible circuit assembly 28. The layers 23 and 27 are thin and flexible, as is the flexible conductive circuit 26, allowing the side segment 22 of the monitor patch 20 between the electrodes 28 and the circuit assembly 24 to bend as shown in FIG. 2A.

FIG. 2C is a functional block diagram 200 illustrating exemplary electronic and sensor components of the monitor patch 20 of FIG. 1 according to certain aspects of the present disclosure. The block diagram 200 shows a processing and sensor interface module 201 and external sensors 232, 234 connected to the module 201. In the illustrated example, the module 201 includes a processor 202, a wireless transceiver 207 having a receiver 206 and a transmitter 209, a memory 210, a first sensor interface 212, a second sensor interface 214, a third sensor interface 216, and an internal sensor 236 connected to the third sensor interface 216. The first and second sensor interfaces 212 and 214 are connected to the first and second external sensors 232, 234 via first and second connection ports 222, 224, respectively. In certain embodiments, some or all of the aforementioned components of the module 201 and other components are mounted on a PCB.

Each of the sensor interfaces 212, 214, 216 can include one or more electronic components that are configured to generate an excitation signal or provide DC power for the sensor that the interface is connected to and/or to condition and digitize a sensor signal from the sensor. For example, the sensor interface can include a signal generator for generating an excitation signal or a voltage regulator for providing power to the sensor. The sensor interface can further include an amplifier for amplifying a sensor signal from the sensor and an analog-to-digital converter for digitizing the amplified sensor signal. The sensor interface can further include a filter (e.g., a low-pass or bandpass filter) for filtering out spurious noises (e.g., a 60 Hz noise pickup).

The processor 202 is configured to send and receive data (e.g., digitized signal or control data) to and from the sensor interfaces 212, 214, 216 via a bus 204, which can be one or more wire traces on the PCB. Although a bus communication topology is used in this embodiment, some or all communication between discrete components can also be implemented as direct links without departing from the scope of the present disclosure. For example, the processor 202 may send data representative of an excitation signal to the sensor excitation signal generator inside the sensor interface and receive data representative of the sensor signal from the sensor interface, over either a bus or direct data links between processor 202 and each of sensor interface 212, 214, and 216.

The processor 202 is also capable of communication with the receiver 206 and the transmitter 209 of the wireless transceiver 207 via the bus 204. For example, the processor 202 using the transmitter and receiver 209, 206 can transmit and receive data to and from the bridge 40. In certain embodiments, the transmitter 209 includes one or more of a RF signal generator (e.g., an oscillator), a modulator (a mixer), and a transmitting antenna; and the receiver 206 includes a demodulator (a mixer) and a receiving antenna which may or may not be the same as the transmitting antenna. In some embodiments, the transmitter 209 may include a digital-to-analog converter configured to receive data from the processor 202 and to generate a base signal; and/or the receiver 206 may include an analog-to-digital converter configured to digitize a demodulated base signal and output a stream of digitized data to the processor 202.

The processor 202 may include a general-purpose processor or a specific-purpose processor for executing instructions and may further include a memory 219, such as a volatile or non-volatile memory, for storing data and/or instructions for software programs. The instructions, which may be stored in a memory 219 and/or 210, may be executed by the processor 202 to control and manage the wireless transceiver 207, the sensor interfaces 212, 214, 216, as well as provide other communication and processing functions.

The processor 202 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

Information, such as program instructions, data representative of sensor readings, preset alarm conditions, threshold limits, may be stored in a computer or processor readable medium such as a memory internal to the processor 202 (e.g., the memory 219) or a memory external to the processor 202 (e.g., the memory 210), such as a Random Access Memory (RAM), a flash memory, a Read Only Memory (ROM), a Programmable Read-Only Memory (PROM), an Erasable PROM (EPROM), registers, a hard disk, a removable disk, or any other suitable storage device.

In certain embodiments, the internal sensor 236 can be one or more sensors configured to measure certain properties of the processing and sensor interface module 201, such as a board temperature sensor thermally coupled to a PCB. In other embodiments, the internal sensor 236 can be one or more sensors configured to measure certain properties of the patient 10, such as a motion sensor (e.g., an accelerometer) for measuring the patient's motion or position with respect to gravity.

The external sensors 232, 234 can include sensors and sensing arrangements that are configured to produce a signal representative of one or more vital signs of the patient to which the monitor patch 20 is attached. For example, the first external sensor 232 can be a set of sensing electrodes that are affixed to an exterior surface of the monitor patch 20 and configured to be in contact with the patient for measuring the patient's respiratory rate, and the second external sensor 234 can include a temperature sensing element (e.g., a thermocouple or a thermistor or resistive thermal device (RTD)) affixed, either directly or via an interposing layer, to skin of the patient 10 for measuring the patient's body temperature. In other embodiments, one or more of the external sensors 232, 234 or one or more additional external sensors can measure other vital signs of the patient, such as blood pressure, pulse rate, or oxygen saturation.

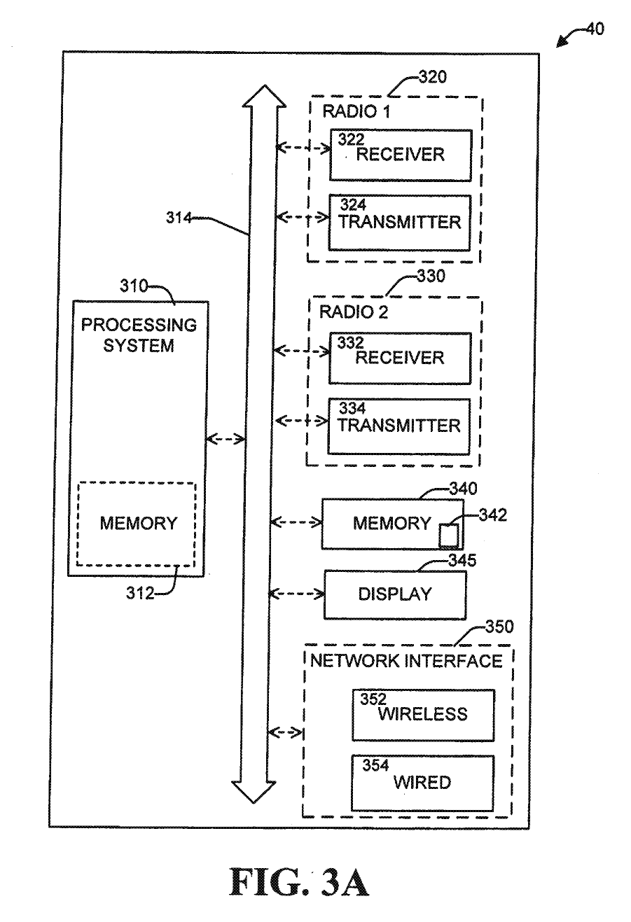

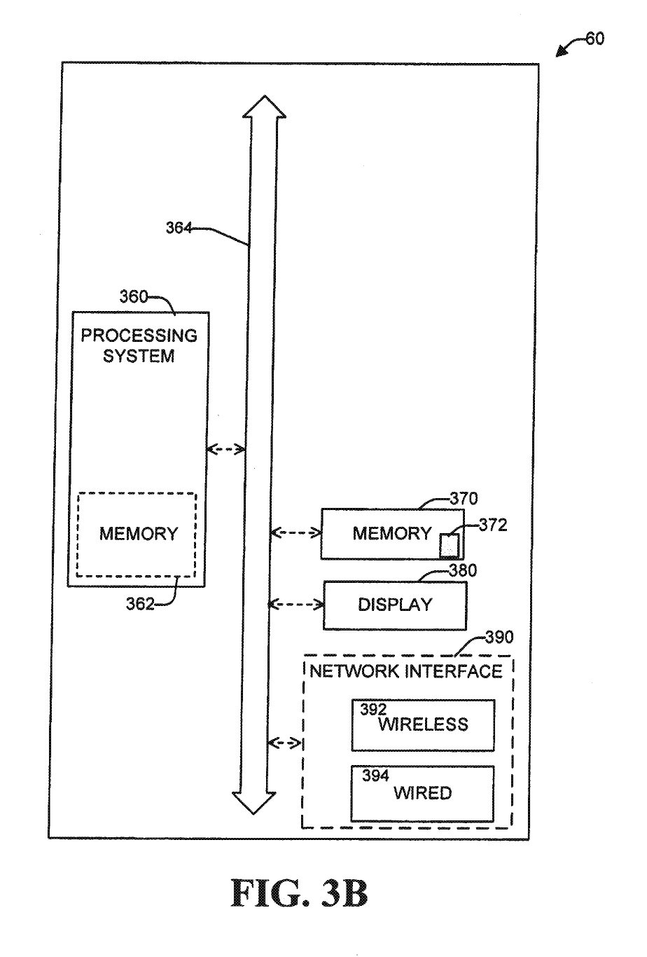

FIG. 3A is a functional block diagram illustrating exemplary electronic components of bridge 40 of FIG. 1 according to one aspect of the subject disclosure. Bridge 40 includes a processor 310, radio 320 having a receiver 322 and a transmitter 324, radio 330 having a receiver 332 and a transmitter 334, memory 340, display 345, and network interface 350 having a wireless interface 352 and a wired interface 354. In some embodiments, some or all of the aforementioned components of module 300 may be integrated into single devices or mounted on PCBs.

Processor 310 is configured to send data to and receive data from receiver 322 and transmitter 324 of radio 320, receiver 332 and transmitter 334 of radio 330 and wireless interface 352 and wired interface 354 of network interface 350 via bus 314. In certain embodiments, transmitters 324 and 334 may include a radio frequency signal generator (oscillator), a modulator, and a transmitting antenna, and the receivers 322 and 332 may include a demodulator and antenna which may or may not be the same as the transmitting antenna of the radio. In some embodiments, transmitters 324 and 334 may include a digital-to-analog converter configured to convert data received from processor 310 and to generate a base signal, while receivers 322 and 332 may include analog-to-digital converters configured to convert a demodulated base signal and sent a digitized data stream to processor 310.

Processor 310 may include a general-purpose processor or a specific-purpose processor for executing instructions and may further include a memory 312, such as a volatile or non-volatile memory, for storing data and/or instructions for software programs. The instructions, which may be stored in memories 312 or 340, may be executed by the processor 310 to control and manage the transceivers 320, 330, and 350 as well as provide other communication and processing functions.

Processor 310 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

Information such as data representative of sensor readings may be stored in memory 312 internal to processor 310 or in memory 340 external to processor 310 which may be a Random Access Memory (RAM), flash memory, Read Only Memory (ROM), Programmable Read Only Memory (PROM), Erasable Programmable Read Only Memory (EPROM), registers, a hard disk, a removable disk, a Solid State Memory (SSD), or any other suitable storage device.

Memory 312 or 340 can also store a list or a database of established communication links and their corresponding characteristics (e.g., signal levels) between the bridge 40 and its related monitor patches 20. In the illustrated example of FIG. 3A, the memory 340 external to the processor 310 includes such a database 342; alternatively, the memory 312 internal to the processor 310 may include such a database.

FIG. 3B is a functional block diagram illustrating exemplary electronic components of server 60 of FIG. 1 according to one aspect of the subject disclosure. Server 60 includes a processor 360, memory 370, display 380, and network interface 390 having a wireless interface 392 and a wired interface 394. Processor 360 may include a general-purpose processor or a specific-purpose processor for executing instructions and may further include a memory 362, such as a volatile or non-volatile memory, for storing data and/or instructions for software programs. The instructions, which may be stored in memories 362 or 370, may be executed by the processor 360 to control and manage the wireless and wired network interfaces 392, 394 as well as provide other communication and processing functions.

Processor 360 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable device or a combination of devices that can perform calculations or other manipulations of information.

Information such as data representative of sensor readings may be stored in memory 362 internal to processor 360 or in memory 370 external to processor 360 which may be a Random Access Memory (RAM), flash memory, Read Only Memory (ROM), Programmable Read Only Memory (PROM), Erasable Programmable Read Only Memory (EPROM), registers, a hard disk, a removable disk, a Solid State Memory (SSD), or any other suitable storage device.

Memory 362 or 370 can also store a database of communication links and their corresponding characteristics (e.g., signal levels) between monitor patches 20 and bridges 40. In the illustrated example of FIG. 3B, the memory 370 external to the processor 360 includes such a database 372; alternatively, the memory 362 internal to the processor 360 may include such a database.

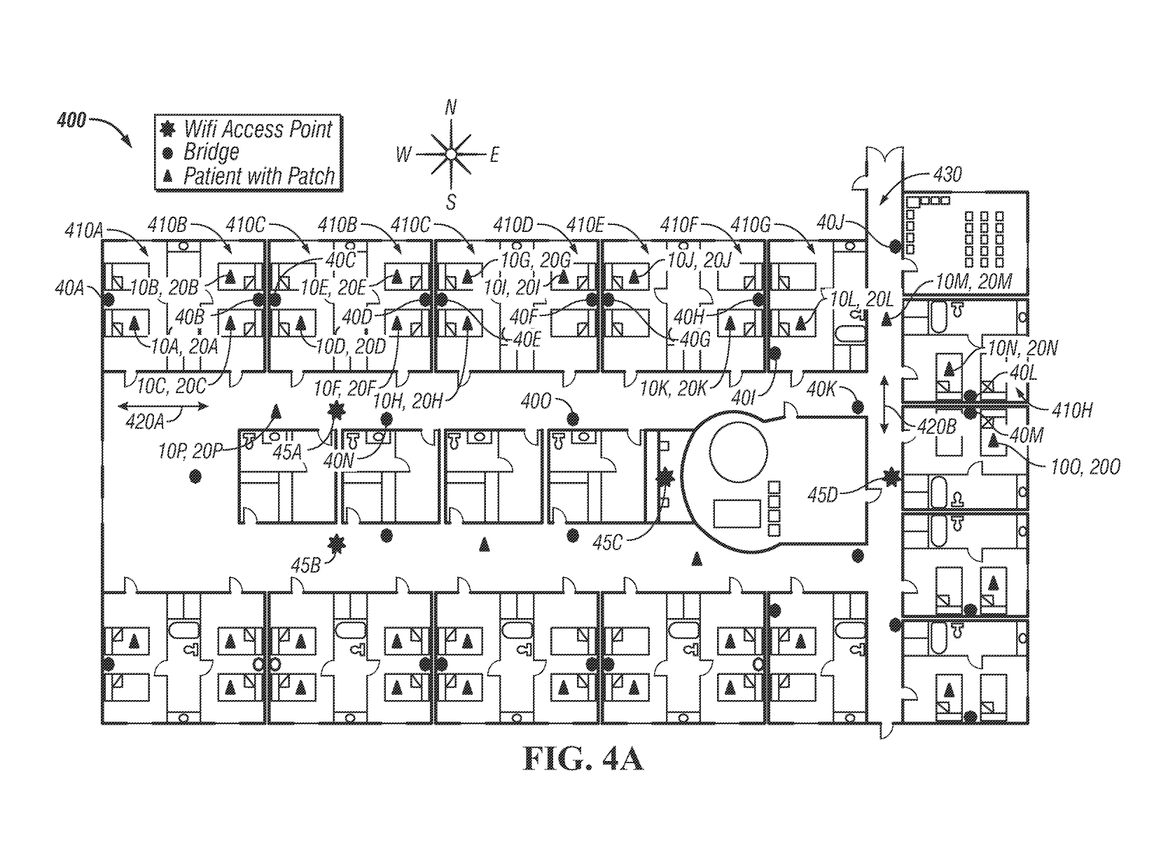

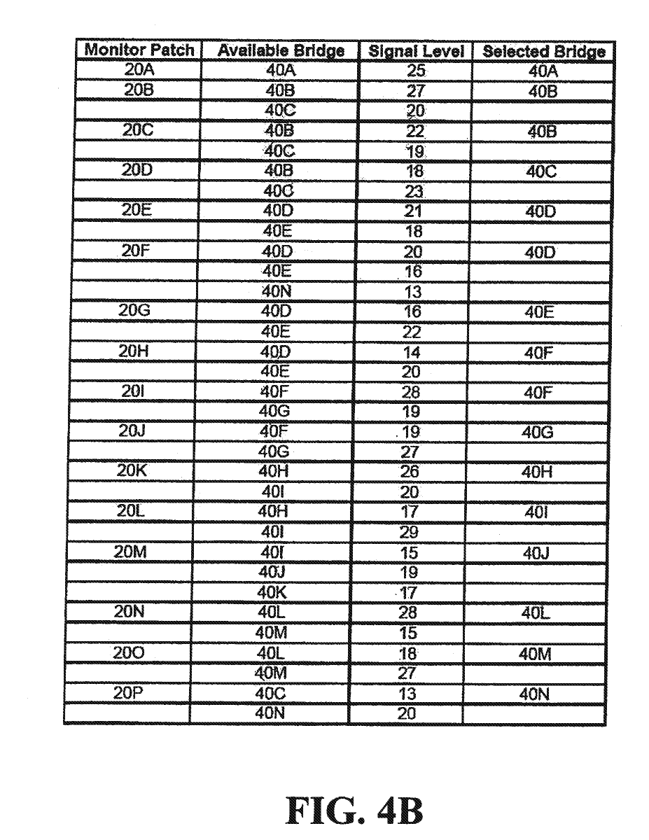

FIG. 4A is a map depicting an exemplary healthcare facility (e.g., a hospital) 400 in which a patient monitoring system such as shown in FIG. 1 is implemented. The healthcare facility 400 includes a plurality of patient rooms 410A-H and hallways 420A, 420B. Shown in the map are a plurality of vital-sign monitor patches 20A-O attached to their respectively assigned patients 10A-O located in the patient rooms 410A-H and hallways 420A, 420B. For ease of illustration and understanding, each patient with attached patch is represented by s triangle in FIG. 4A. The facility 400 also includes a plurality of bridges 40A-O located at specified locations in the facility 400 and configured to engage in wireless communication with the monitor patches 20A-O. The bridges 40A-O are represented has circle icons. Although there are other patient rooms, bridges, and patients/monitor patches shown in the map, for the sake of simplicity, the following description will focus on the patient rooms 410A-H, bridges 40A-O and patients/monitor patches 10A-O/20A-O. In the illustrated example, the bridges 40A-O are in turn connected to WiFi access points 45A-D (shown as “stars”) that are configured to route data between the bridges 40A-O and a surveillance server 60 (FIG. 1).

Preferably, a bridge 40 is selected for each monitor patch 20 through which the monitor patch 20 sends and receives signals to and from the server 60 via an access point 45A,B,C, or D. For example, the monitor patch 20B worn by the patient 10B in the room 41013 wirelessly transmits one or more signals comprising information indicative of his vital signs (e.g., heart rate) to the bridge 40B. The bridge 40B receives the signals and sends the information extracted from the signals to the access point 45 as data via either a wired or wireless connection. The access point 45A sends the data to the surveillance server 60 via either a wired or wireless connection. As other examples, the monitor patch 20A worn by the patient 10A in the room 410A sends data to the server 60 via the bridge 60A and the access point 45A; the monitor patch 20P worn by the patient 10P, walking eastward in the hallway 420A, sends data to the server 60 via the bridge 40N and the access point 45A; and the monitor patch 20M worn by the patient 10M, walking southward in the hallway 420B, sends data to the server 60 via the bridge 40J and the access point 45D.

Because a monitor patch 20 and a bridge 40 have limited wireless ranges, the monitor patch 20 is located in close proximity from the bridge 40 with which the monitor patch has a communicative association. Therefore, it is possible to track the location of a monitor patch 20 by knowing the location of the selected bridge 40. In certain embodiments of a monitoring network of the present disclosure, the surveillance server 60 is configured to track locations of the monitor patches 20A-O by maintaining a database comprising information indicative of monitor patches 20A-O and their selected bridges 40A-O. In certain embodiments, the database further comprises a list of unselected but linkable bridges with which each of the monitor patches 20A-O is capable of engaging in a bidirectional wireless data communication.

FIG. 4B is a portion of an exemplary database comprising the monitor patches 20A-20P (first column), their linkable bridges (second column), signal levels associated with the communication links between the monitor patches and the linkable bridges (third column) in an arbitrary unit (e.g., dbm), and selected bridges (fourth column) according to certain aspects of the present disclosure. Such a database may be stored in the memory 370 (FIG. 3B) of the server 60 as database 372, for example. Alternatively, the database may be stored in a memory located outside the server 60 (e.g., on a network) but accessible by the server 60 via, e.g., a wired or wireless interface 992, 994. For ease of illustration, it is assumed that the database of FIG. 4B corresponds to the database 372 of FIG. 3B.

For example, the database 372 shows the monitor patch 20A having one linkable bridge 40A which is also the selected bridge. The database 372 also shows the monitor patches 20B and 20C having the same linkable bridges 40B, 40C of which the bridges 40B is the selected bridge for the both monitor patches. The database 372 also shows the monitor patch 20D having the same linkable bridges 40B, 40C of which the bridge 40C is the selected bridge. The above portions of the database 372 relating to the monitor patches 20B-D reflect the fact that the monitor patches 20B, 20C are in the room 410B having the bridge 40B located therein, while the monitor patch 20D is in the room 410C having the bridge 40C located therein. Therefore, while the bridge 40B is capable of communicating with the monitor patch 20D due to their close proximity, the bridge 40C is selected for the monitor patch 20D, e.g., by the server 60, due to the bridge's closer proximity to the monitor patch 20D. The database 372 also shows the monitor patch 20M worn by the patient 10M having three linkable bridges 40I, 40J, 40K of which the bridge 40J is the currently selected bridge; and the monitor patch 20P worn by the patient 10P having two linkable bridges 40C, 40N of which the bridge 40N is the currently selected bridge.

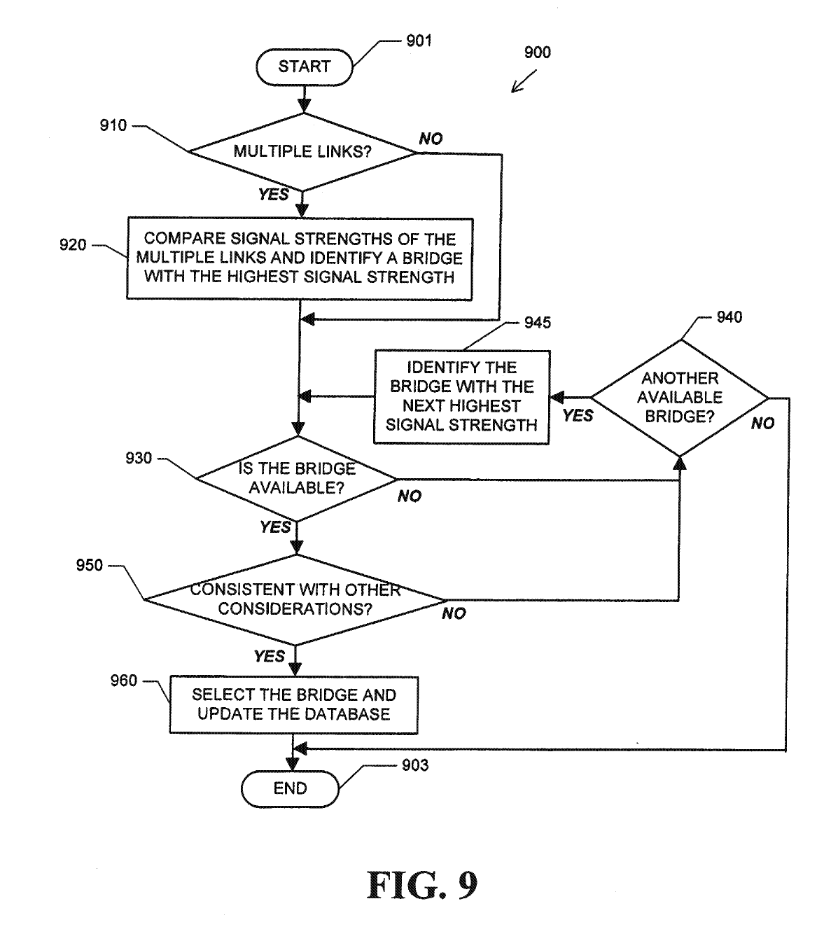

The signal levels (third column) associated with various bridge-patch communication links in the database 372 represent the strengths of wireless signals (e.g., acknowledgment signals) from the bridges 40A-O received by the monitor patches 20A-O. As will be described below with respect to FIG. 9, the signal levels can be used for a bridge selection by the surveillance server 60.

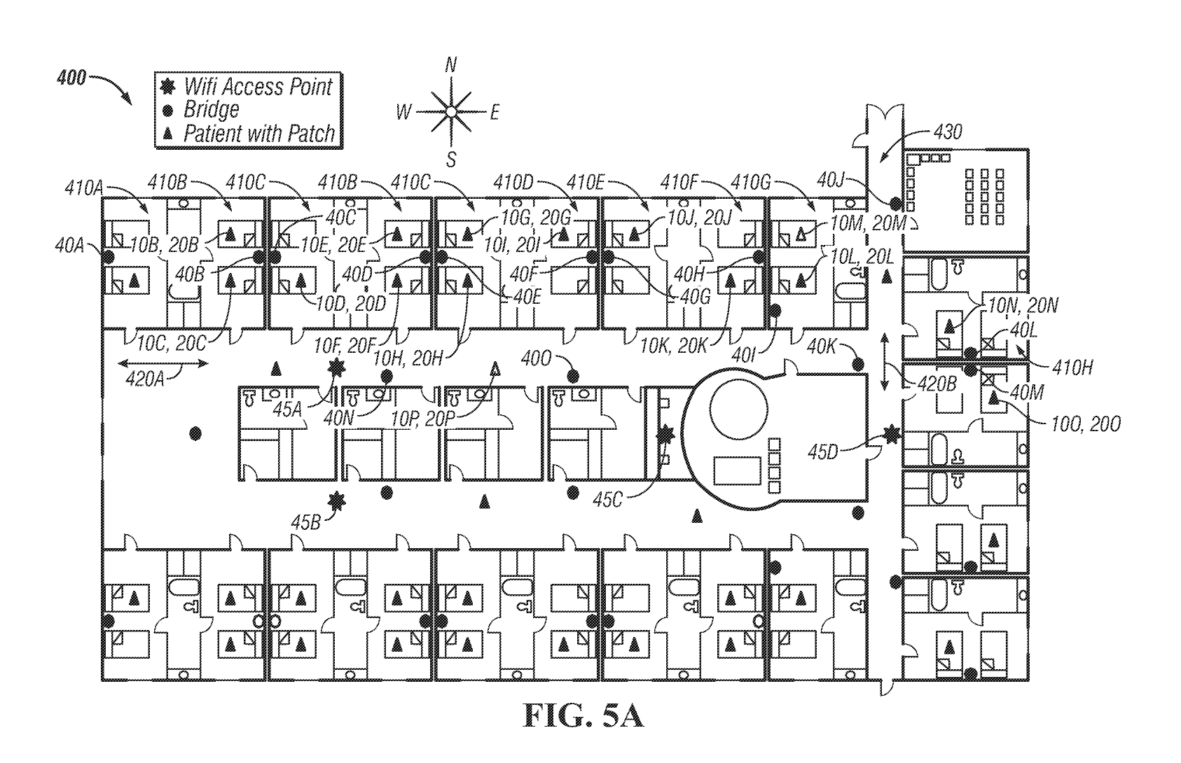

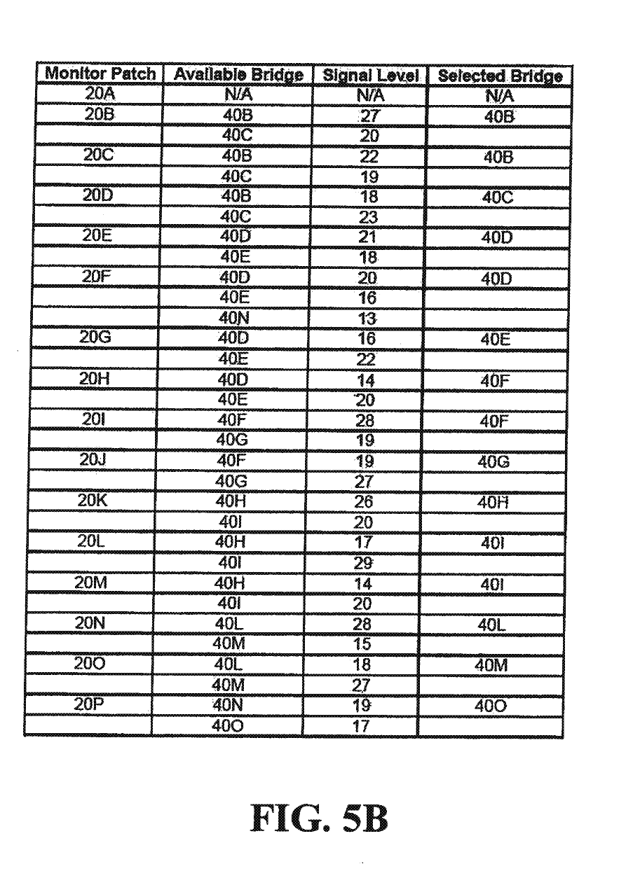

FIG. 5A is a map of the exemplary healthcare facility 400 depicted in FIG. 4A after a passage of time from the map of FIG. 4A. The map of FIG. 4B is the same as the map of FIG. 4A except for the following changes:
1) The patient 10A wearing the monitor patch 20A has left the facility 400.
2) The patient 10M wearing the monitor patch 20M has now returned to her patient room 410G.
3) The patient 10P wearing the monitor patch 20P and walking along the hallway 420A has now progressed to the middle of the hallway 420A.

In response to the changes, the surveillance server 60 has updated the database 372 discussed above with respect to FIG. 4B. FIG. 5B is a portion of an updated version of the database 372 shown in FIG. 4B according to certain embodiments of the present disclosure. The updated database 372 shows the monitor patch 20A having neither a linkable bridge nor a selected bridge, reflecting the fact that the monitor patch 20A is longer in communication range of any of the bridges in the monitoring system. The updated database also shows the monitor patch 20M now having three linkable bridges 40H, 40I, 40K of which the bridge 40I is the currently selected bridge, reflecting the fact that the monitor patch 20M is now in the room 410G having the bridge 40I located therein. The updated database also shows the monitor patch 20P having two linkable bridges 40N, 40O of which the bridge 40O is the currently selected bridge. In the illustrated example of FIG. 5A, the monitor patch 20P is substantially equidistant from both the bridge 40N and the bridge 40O and the respective signal strengths are similar (19 versus 17). Hence, the monitor patch 20P can be served equally well by both bridges 40N, 40O. Notwithstanding the fact that the signal strength associated with the bridge 40N is slightly higher than that associated with the bridge 40O, control software in the surveillance server 60 has selected the bridge 40O based on the consideration that the monitor patch 20P worn by the patient 10P has been moving towards the bridge 40O and away from the bridge 40N and, hence, is likely to be served longer by the former bridge 40O than by the latter bridge 40N.

Henceforth, specific reference numbers (e.g., bridge 40A, monitor patch 20C) will be used when referring to specific devices, while generic references (bridge 40, monitor patch 20) will be used when referring to devices in a general sense.

A communication link between a bridge 40 and a monitor patch associated 20 associated with a patient can be considered established, for example, when the bridge 40 has received one or more regularly transmitted signals (e.g., those indicative of vital signs of the patient) from the monitor patch 20 or when the bridge 40 has received an acknowledgment signal from the bridge 40 in response to a query signal sent out via the bridge 40. From the perspective of the monitor patches 20, the bridge is one of linkable bridges for the monitor patches 20. Conversely, an established communication link between a bridge 40 and a monitor patch 20 can be considered lost when the bridge 40 can no longer receive regularly transmitted signals from the monitor patch 20 or when the bridge 40 does not receive an acknowledgment signal from the monitor patch 20 in response to a query signal.

In certain embodiments, upon occurrence of a new communication link or loss of an existing communication link, the bridge 40 automatically sends the message to the surveillance server 60 (FIG. 1), which, in turn, updates the database 372 stored in memory (e.g., 370) associated with the server 60, such as a hard disk or an external data storage device accessible by the server. In some embodiments, each of the bridges 40A-O includes memory (e.g., 312, 340 of FIG. 3A) for storing a list or database 342 of monitor patches 20A-O with which the bridge 40 has established communication links. A processor (e.g., 310 of FIG. 3A) executing control software in the bridge 40 can update the list 342 stored in the memory (e.g., 312, 340) of the bridge 40 to keep the list current. The processor 310 then sends the updated list 342 or a portion thereof to the surveillance server 60.

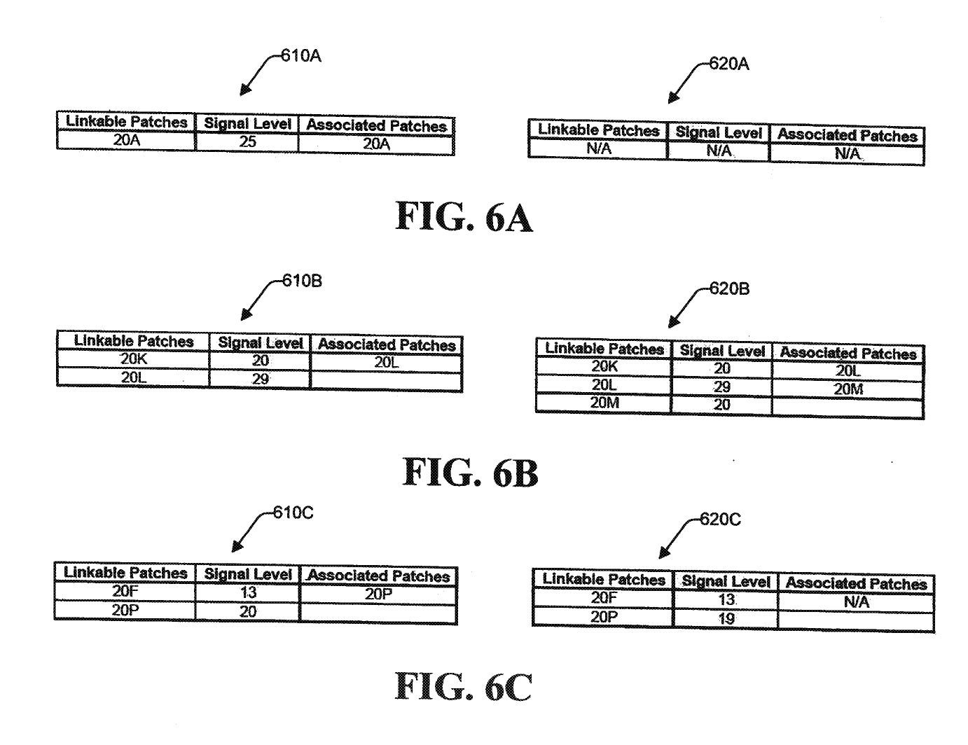

FIG. 6A shows a first list 610A stored in the bridge 40A at the time of FIG. 4A, and a second list 620A which corresponds to an updated list stored in the bridge 40A at the time of FIG. 5A. The first column of the first list 610A enumerates linkable patches corresponding to all monitor patches with which the bridge 40A has established communication links at the time of FIG. 4A. The second column of the first list 610A enumerates associated patches corresponding to all monitor patches 20 for which the bridge 40A has been selected for communicative association at the time of FIG. 4A. As can be seen from FIG. 4A and correspondingly reflected in the first list 610A, the bridge 40A has an established communication link only with the monitor patch 20A. The bridge 40A is also the selected bridge 40 for the monitor patch 20A, or, conversely, the monitor patch 20A is an associated patch for the bridge 40A. As can be seen from FIG. 5A and correspondingly reflected in the second list 620A, the bridge 40A has neither a linkable patch nor an associated patch at the time of FIG. 5A, reflecting the fact that between the time of FIG. 4A and the time of FIG. 5A, the patient 10A wearing the monitor patch 20A has left the healthcare facility 400, or the patch 20A has been removed and deactivated.

FIG. 6B shows a first list 610B stored in the bridge 40I at the time of FIG. 4A, and a second list 620B that corresponds to an updated list stored in the bridge 40I at the time of FIG. 5A. As can be seen from FIG. 4A and correspondingly reflected in the first list 610B, the bridge 40A has established communication links with the monitor patches 20K and 20L of which the monitor patch 20L is the associated patch. As can be seen from FIG. 5A and correspondingly reflected in the second list 620B, the bridge 40I has established communication links with the monitor patches 20K, 20L, and 20M of which the monitor patches 20L and 20M are the associated patches at the time of FIG. 5A, reflecting the fact that between the time of FIG. 4A and the time of FIG. 5A, the patient 10M wearing the monitor patch 20M has entered the patient room 410G.

FIG. 6C shows a first list 610C stored in the bridge 40N at the time of FIG. 4A, and a second list 620C which corresponds to an updated list stored in the bridge 40N at the time of FIG. 5A. As can be seen from FIG. 4A and correspondingly reflected in the first list 610B, the bridge 40N has established communication links with the monitor patches 20F and 20P of which the monitor patch 20P is the associated patch. As can be seen from FIG. 5A and correspondingly reflected in the second list 620B, the bridge 40N has established communication links with the monitor patches 20, but has no associated patch, reflecting the condition that between the time of FIG. 4A and the time of FIG. 5A, the patient 10P wearing the monitor patch 20P has progressed to the center of the hallway 420A towards the bridge 40O and the surveillance server 60 has selected the bridge 40O for the monitor patch 20P as discussed above with respect to FIG. 5B.

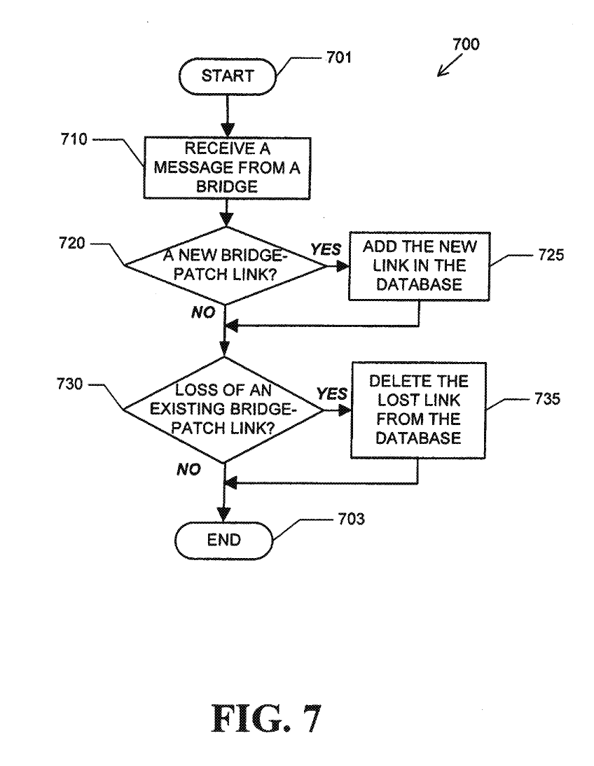

FIG. 7 is a flowchart illustrating a process 700 for tracking locations of monitor patches 20 by keeping and updating a database comprising information indicative of the monitor patches 20 and their linkable and selected bridges 40 according to certain aspects of the present disclosure. For the purposes of illustration only, without any intent to limit the scope of the present disclosure in any way, the process 700 will be described with reference to FIGS. 1, 4A-B, and 5A-B. The process 700 begins at start state 701 and proceeds to operation 710 in which a surveillance server 60 receives a message from a bridge 40 indicating that the bridge 40 has established a new communication link with a monitor patch 20 or lost an existing communication link with a monitor patch 20 or both. For example, if the message were sent from the bridge 40A of FIGS. 4A and 5A, the message would indicate loss of an existing communication link with the monitor patch 20A. On the other hand, if the message were sent from the bridge 40I, the message would indicate a new (previously unavailable) communication link with the monitor patch 20M.

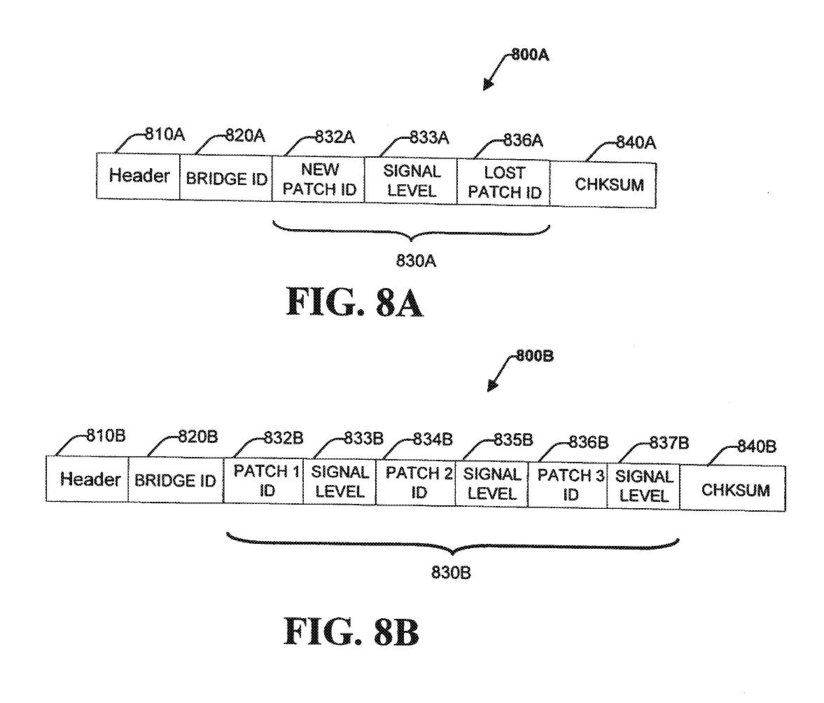

FIG. 8A is a diagram illustrating an exemplary data structure for a message 800A indicating a new communication link and/or loss of an existing communication link according to certain aspects of the present disclosure. In the illustrated example, the message 800A includes a header field 810A for storing a message header, an ID field 820A for storing an ID for a bridge sending the message, a data field 830A for storing information relating to a new communication link and/or loss of an existing communication link, and optionally a field 840 for storing a checksum. The header field 810A can include subfields for indicating a total number of monitor patches with which the bridge has established new communication links and a total number of monitor patches with which the bridge has lost existing communication links. The data field 830A includes a first subfield 832A for storing an ID for a monitor patch that the bridge has established a new communication link, a second subfield 833A for storing data indicative of a signal level or strength associated with the new communication link, and a third subfield 836A for storing an ID for a monitor patch with which the bridge has lost an existing communication link. If the message 800A were sent from the bridge 40A while transitioning from the configuration of FIG. 4A to the configuration of FIG. 5A, the ID field 820 would include data indicative of the bridge 40A, and the third subfield 836A would include data indicative of the monitor patch 20A. The message embodiment shown in FIG. 8A is exemplary only, as other message embodiments may be employed. The surveillance server 60 upon receiving the message 830A can update the database such as the one shown in FIG. 4B as further described below.

FIG. 8B is a diagram illustrating an exemplary data structure for an alternative message 800B indicating a new communication link and/or loss of an existing communication link according to alternative aspects of the present disclosure. In the illustrated example, the message 820B includes a header field 810B for storing a message header, an ID field 820B for storing an ID for a bridge sending the message, a data field 830B for storing information relating to all monitor patches with which the bridge has established communication links, and a field 840B for storing a checksum. The header field 810A can include subfields for indicating a total number of linkable monitor patches 20 with which the bridge 40 has established communication links. The data field 830B includes a first subfield 8328 for storing an ID of a first linkable monitor patch 20, a second subfield 833B for storing data indicative of a signal strength or level associated with the communication link between the bridge 40 and the first linkable monitor patch 20. The data field 830B includes other subfields 834B, 835B, 836B, 837B for storing ID's and data indicative signal strengths for additional linkable monitor patches 20. If the message were sent from the bridge 40I in the configuration of FIG. 5A (corresponding to the list 620B of FIG. 6B), the data fields 832B, 834B, 836B would include data indicative of the monitor patches 20K, 20L, 20M, respectively, for example. In certain embodiments, each of the subfields 832B, 834B, 836B includes a single bit for indicating whether the monitor patch 20 indicated by the subfield is associated with the bridge 40 or not (i.e., whether the bridge 40 is the selected bridge 40 for the monitor patch 20), In those embodiments, such a bit would be clear for the subfield 832B (for the monitor patch 20K), but set for the subfields 834B, 836B (for the monitor patches 20L, 20M). The message embodiment of FIG. 8A is exemplary only, as other message embodiments may be employed. The surveillance server 60, upon receiving the message 830B, can update the database such as the one shown in FIG. 4B as further described below.

Returning to FIG. 7, the process 700 proceeds to decision state 720 in which it is determined whether the received message indicates that the bridge 40 sending the message has established at least one new communication link with a monitor patch 20, e.g., by starting to receive regularly transmitted signals from the monitor patch. In case of the message 800A of FIG. 8A, this determination can involve control software running in a processor of the surveillance server 60 searching for nonzero data in the data field 832A (“NEW PATCH ID”). In case of the message 800B of FIG. 8B, this determination can involve the control software comparing the linkable monitor patches 20 indicated in the data field 830B of the message 800B to previously stored linkable monitor patches 20 for the bridge 40 in order to discover one or more monitor patches 20 that are newly present in the message. If it is determined at the state 720 that no new communication link has been established for the bridge 40 (NO), the process 700 proceeds to decision state 730 to be described below.

On the other hand, if it is determined at the decision state 720 that at least one new communication link has been established for the bridge 40 (YES), the process 700 proceeds to operation 725 in which database 372 comprising information indicative of communication links between monitor patches 20A-O and bridges 40A-O is accessed and the new communication link is added to the database. Examples of such additions include the communication links between the monitor patch 20M and the bridge 40I and the communication link between the monitor patch 20P and the bridge 40O, both of which are not present in the database shown in FIG. 4A but present in the updated database shown in FIG. 5B. The process 700 then proceeds to decision state 730 described below.

In the decision state 730 it is determined whether the received message indicates that the bridge 40 sending the message has lost at least one existing communication link with a monitor patch 20, e.g., by failing to receive regularly transmitted signals from the monitor patch 20. In case of the message 800A of FIG. 8A, this determination can involve control software running in a processor of the surveillance server 60 looking for nonzero data in the subfield 836A. In case of the message 800B of FIG. 8B, this determination can involve the control software comparing the linkable monitor patches 20 indicated in the data field 830B of the message 800B to previously stored linkable monitor patches 20 for the bridge 40 in order to discover one or more monitor patches 20 that are not longer present in the message.

If it is determined at the decision state 730 that no existing communication link has been lost for the bridge 40 (NO), the process 700 ends at state 703. On the other hand, if it is determined at the decision state 730 that at least one existing communication link has been lost for the bridge (YES), the process 700 proceeds to operation 725 in which a database comprising information indicative of communication links between monitor patches 20A-O and bridges 40A-O is accessed and the communication link is deleted from the database. Examples of such deletion include the communication link between the monitor patch 20M and the bridge 40J and the communication link between the monitor patch 20P and the bridge 40C, which is present in the database shown in FIG. 5A but not present in the updated database shown in FIG. 5B. The process 700 ends at state 703.

Therefore, the surveillance server 60, by maintaining and updating a database of bridge-patch communication links using a process such as the process 700 based on messages received from bridges 40, can track locations of monitor patches 20A-O in the healthcare facility 400. In certain embodiments, the surveillance server 60, after receiving one or more of such messages or at scheduled intervals, can select a particular bridge 40 among a set of linkable bridges 40 for a particular monitor patch 20. After such a bridge selection, in certain embodiments, the server 60 prevents other linkable but unselected bridges 40 from communicating with the particular monitor patch 20.

FIG. 9 is a flowchart illustrating an exemplary process 900 for a bridge selection process according to certain aspects of the present disclosure. The process 900 begins at start state 901 and proceeds to decision state 910 in which it is determined whether there are multiple bridge-patch communication links (e.g., multiple linkable bridges) available for a particular monitor patch 20. If it is determined at the decision state 910 that there is only one communication link (e.g., one linkable bridge) available for the monitor patch, the process 900 proceeds to another decision state 930 to be described below. On the other hand, if it is determined at the decision state 910 that there are multiple communication links (e.g., multiple linkable bridges 40) for the monitor patch 20, the process 900 proceeds to operation 920 in which signal strengths of the multiple communication links are compared, and a bridge 40 associated with the highest signal strength is identified. For example, in case of the monitor patch 20M in the configuration of FIG. 4B, the communication link between the monitor patch 20M and the bridge 40J has the highest signal strength (19) among all available communication links.

The process 900 proceeds to decision state 930 in which it is determined whether the bridge 40 being considered for selection (e.g., the only bridge in case of one communication link or the identified bridge in case of multiple communication links) is available for communication with the monitor patch 20. This determination can involve determining by the surveillance server 60 or by the bridge 40 the number of monitor patches 20 with which the bridge 40 is currently associated (e.g., the number of monitor patches to which the bridge is currently the selected bridge) in order to determine whether the bridge 40 is currently overloaded. If it is determined at the decision state 930 that the bridge 40 being considered for selection is not available (NO), the process 900 proceeds to decision state 940 in which it is determined whether there are one or more other linkable bridges 40 with which the monitor patch 20 can be associated. If it is determined at the decision state 940 that there is no other linkable bridge 40 (NO), the process 900 ends at state 903. On the other hand, if it is determined at the decision state 940 that there are one or more other linkable bridges 40 (YES), the process 900 proceeds to operation 945 in which another linkable bridge 40 associated with the next highest signal strength is identified, and then back to the decision state 930 for determining availability of the other bridge 40.

On the other hand, if it is determined at the decision state 930 that the bridge 40 being considered for selection is available (YES), the process 900 proceeds to decision state 950 in which it is determined whether the selection of the bridge 40 being considered is consistent with other considerations. For example, as indicated above with respect to FIG. 5B, control software running in the surveillance server 60 selected the bridge 40O over the bridge 40N in spite of the condition that the signal strength associated with the bridge 40N is currently stronger than the signal strength associated with the bridge 40O. The selection is based on the additional consideration that the patch 20P has been moving away from the bridge 40N and towards the bridge 40O.

If it is determined at the decision state 930 that the selection of the bridge 40 is not consistent with other considerations (NO), the process 900 proceeds to the decision state 940 and to the operation 945 and back to the decision state 930 as discussed above. On the other hand, if it is determined at the decision state 930 that the selection of the bridge 40 is consistent with other considerations (YES), the process 900 proceeds to operation 960 in which the bridge 40 is selected for the monitor patch 20 and, the database of bridge-patch communication links is updated to reflect the new selection. The process 900 ends at state 903.

At times, a bridge 40 can lose power or break down or otherwise become inoperable and can no longer carry data between associated monitor patches 20 and the surveillance server 60. For example, if the bridge 40I becomes inoperable, the monitor patches 20L and 20M can no longer send data to the surveillance server 60 via the bridge 40I. In certain embodiments, the surveillance server 60, upon recognition of such an occurrence, selects alternative bridges 40 for the monitor patches 20 so as to route data between the monitor patches 20 and the surveillance server 60 via the alternative bridges 40.

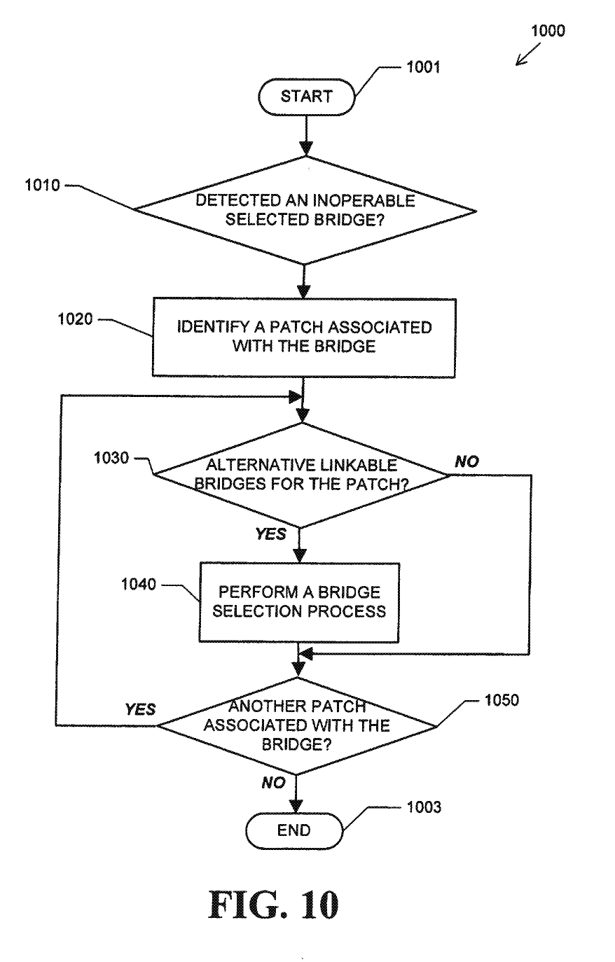

FIG. 10 is a flowchart illustrating a process 1000 for detecting an inoperable bridge 40 and selecting an alternative bridge 40 to replace the inoperable bridge 40 for the monitor patches 20 that were previously associated with the inoperable bridge 40 according to certain aspects of the present disclosure. The process 1000 begins at start state 1001 and proceeds to decision state 1010 in which it is determined whether an inoperable selected bridge 40 has been detected. The determination can include failing to receive regularly transmitted messages from the selected bridge 40 or failing to receive an acknowledgment message from the bridge 40 in response to a query message sent to the bridge 40 by the surveillance server 60. If it is determined at the decision state 1010 that an inoperable selected bridge 40 has not been detected (NO), the process 1000 loops back to the decision state 1010 to await such an occurrence. On the other hand, if an inoperable selected bridge 40 has been detected (YES), the process 1000 proceeds to operation 1020 in which a monitor patch 20 associated with the selected bridge 40 is identified from, e.g., database (e.g., 372) such as the ones shown in FIGS. 4B and 5B. For example, if the selected bridge found to be inoperable is the bridge 40D, the monitor patch 20E can be identified. After the identification, the process proceeds to decision state 1030.

In the decision state 1030, it is determined whether there are one or more alternative linkable bridges 40 for the identified monitor patch from, e.g., a list in a database such as the ones shown in FIGS. 4B and 5B. If it is determined at the decision state 1030 that there is no alternative bridge 40 (NO), the process 1000 proceeds to decision state 1050 which will be described below. On the other hand, if it is determined at the decision state 1030 that there are one or more alternative linkable bridges 40 for the identified monitor patch (YES) (the bridge 40B for the monitor patch 20E in the above example), the process 1000 proceeds to operation 1040 in which a bridge selection process such as the one described above with respect to FIG. 9 is performed in order to select an alternative bridge 40 for the identified monitor patch 20,

The process 1000 then proceeds to decision state 1050 in which it is determined whether there is another monitor patch 20 associated with the selected bridge 40 determined to be inoperable at the decision state 1010. If it is determined at the decision state 1050 that there is no other monitor patch 20 associated with the inoperable bridge 40, the operation 1000 ends at state 1003. On the other hand, if it is determined at the decision state 1050 that there is another monitor patch 20 associated with the inoperable bridge 40 (YES) (the monitor patch 20E for the bridge 40D in the above example), the process 1000 loops back to the decision state 1030 in which it is determined where there are one or more alternative bridges 40 for the other monitor patch 20 and then to the selection operation 1040 and decision state 1050. The loop is repeated until it is determined at the decision state 1050 that there is no other monitor patch 20 associated with the inoperable bridge 40 in which case the process 1000 ends at state 1003.

In certain aspects, the knowledge of locations of monitor patches (e.g., 20A-O of FIGS. 4A and 4B) can be used for tracking patients (e.g., 10A-O) wearing the monitor patches 20 in a healthcare facility (e.g., hospital). As discussed above with respect to FIG. 7, a surveillance server (e.g., 60 of FIG. 1) can track locations of monitor patches 20 by keeping and updating database 372 comprising information indicative of the monitor patches 20 and their linkable and selected bridges 40 based on messages received from various bridges 40. Therefore, assuming that the locations of various bridges 40 in the facility and the names of patients 10 to whom the monitor patches 20 are assigned are known, locations of the patients 10 can also be tracked.

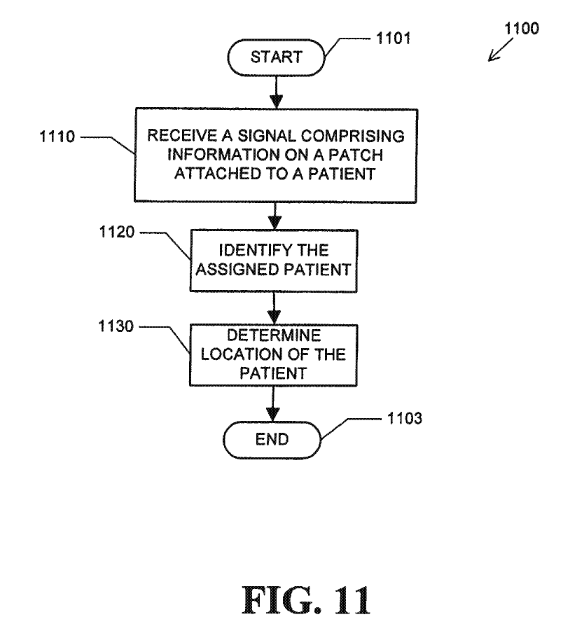

FIG. 11 is a flowchart illustrating a process 1100 for determining locations of patients 10 in a healthcare facility according to certain aspects of the present disclosure. The process 1100 begins at start state 1101 and proceeds to operation 1110 in which a surveillance server 60 receives a signal comprising information relating to a monitor patch 20 attached to a patient 10 from a selected bridge 40 for the monitor patch 20. The signal can be, for example, one of the messages 800A and 800B discussed above with respect to FIGS. 8A and 8B. After receiving the signal, surveillance server 60 can update database 372 such as the ones shown in FIGS. 4A and 4B as discussed above with respect to FIG. 7. In certain embodiments, the signal is generated by the bridge 40 in response to a newly established communication link between the bridge 40 and the monitor patch 20 trigged by the patient 10 being moved into her new patient room. In other embodiments, the signal is generated by the bridge 40 in response to a query signal sent to the bridge 40 by the surveillance server 60.

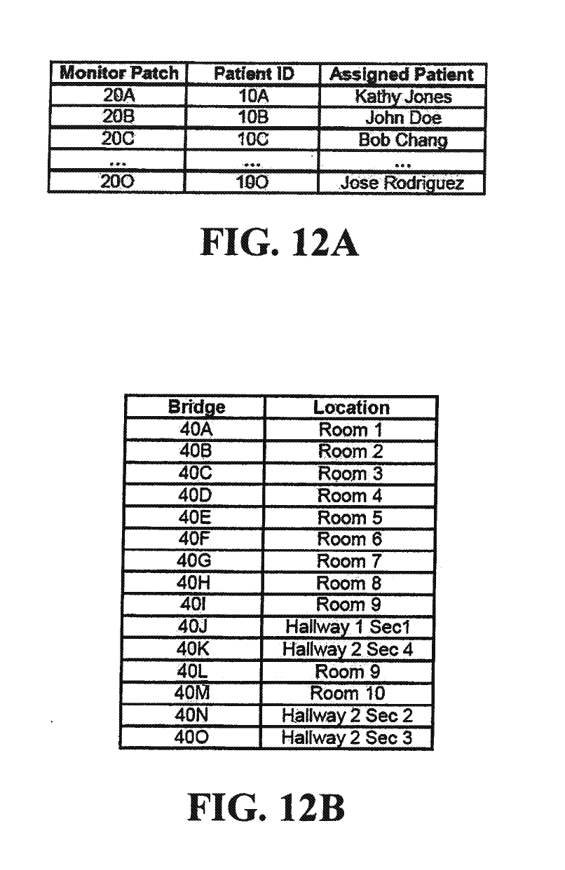

The process 1100 proceeds to operation 1120 in which a patient to whom the monitor patch 20 is attached is identified. In certain embodiments, the identification operation includes control software running in the surveillance server 60 accessing a database such as the one shown in FIG. 12A that lists monitor patches 20 (first column) and their assigned patients 10 (second column). In other embodiments, the received signal includes information indicative of the patient 10 (e.g., the patient ID), and the control software extracts the information from the signal. The process 1100 proceeds to operation 1130 in which location of the patient 10 is determined. In certain embodiments, the operation 1130 includes the control software accessing a database such as the one shown in FIG. 12B that lists locations of various bridges 40 in the facility. In other embodiments, the received signal includes information indicative of the location of the bridge 40 that sent the signal, and the control software extracts the information from the signal. In some embodiments, the determined location (e.g., “Room 3”) is displayed on a display associated with a hospital system (e.g., the workstation 100 of FIG. 1). Alternatively, the display can graphically indicate the patient location on a hospital map such as the ones shown FIGS. 4A and 4B.

The foregoing description is provided to enable any person skilled in the art to practice the various embodiments described herein. While the foregoing embodiments have been particularly described with reference to the various figures and embodiments, it should be understood that these are for illustration purposes only and should not be taken as limiting the scope of the claims.

The word “exemplary” is used herein to mean “serving as an example or illustration.” Any aspect or design described herein as “exemplary” is not necessarily to be construed as preferred or advantageous over other aspects or designs.

A reference to an element in the singular is not intended to mean “one and only one” unless specifically stated, but rather “one or more.” The term “some” refers to one or more. Underlined and/or italicized headings and subheadings are used for convenience only, do not limit the invention, and are not referred to in connection with the interpretation of the description of the invention. All structural and functional equivalents to the elements of the various embodiments of the invention described throughout this disclosure that are known or later come to be known to those of ordinary skill in the art are expressly incorporated herein by reference and intended to be encompassed by the invention. Moreover, nothing disclosed herein is intended to be dedicated to the public regardless of whether such disclosure is explicitly recited in the above description.

What is claimed is:

1. A method of tracking vital-sign monitor patches in a vital-sign monitoring network, the method comprising:
identifying at least one of a new wireless communication link and loss of an existing wireless communication link between a vital-sign monitor patch and a bridge in the monitoring network;
accessing a database comprising information indicative of wireless communication links between vital-sign monitor patches and bridges in the monitoring network; and
updating the information in the database to indicate the new wireless communication link or the loss of the existing wireless communication link.

2. The method of claim 1 wherein the vital-sign monitor patch is configured to monitor one or more of vital signs of a person to whom the monitor patch is attached.

3. The method of claim 1, wherein identifying the new wireless communication link comprises receiving a message from the bridge indicating that the bridge has established the new wireless communication link with the vital-sign monitor patch.

4. The method of claim 1, wherein identifying the loss of an existing wireless communication link comprises receiving a message from the bridge indicating that the bridge has lost the existing wireless communication link with the vital-sign monitor patch.

5. A vital-sign monitoring system, comprising:
a plurality of vital-sign monitor patches configured to monitor one or more vital signs of patients to whom the vital-sign monitor patches are attached;
a surveillance server configured to gather data relating to the one or more vital signs of the patients from the plurality of vital-sign monitor patches;
a plurality of bridges configured to provide data connections between the plurality of vital-sign monitor patches and the surveillance server; and
a database configured to store information indicative of wireless communication links between at least some of the plurality of vital-sign monitor patches and at least some of the plurality of bridges,
wherein the database is updated to indicate a new wireless communication link or loss of an existing wireless communication link between a vital-sign monitor patch and a bridge.

6. The monitoring network of claim 5, wherein the one or more vital-signs include at least one of body temperature, pulse rate, blood pressure, and respiratory rate.

7. The system of claim 5, wherein the database is updated by the bridge after the bridge has established the new wireless communication link with the vital-signs patch.

8. The system of claim 5, wherein the database is updated by the bridge after the bridge has lost an existing wireless communication link.

9. The system of claim 5, wherein at least one bridge among the plurality of bridges include a memory for storing a list of vital-sign monitor patches with which the at least one bridge has established a wireless communication link.

10. The system of claim 5, wherein the database is updated by the surveillance server after receiving a message from the bridge indicating that the bridge has established the new wireless communication link with the vital-sign monitor patch.

11. The system of claim 5, wherein the database is updated by the surveillance server after receiving a message from the bridge indicating that the bridge has lost the existing wireless communication link with the vital-sign monitor patch.

12. The system of claim 5, wherein at least one vital-sign monitor patch among the plurality of vital-sign monitor patches has a plurality of wireless communication links with two or more bridges among the plurality of bridges.

13. The system of claim 12, wherein the surveillance server is further configured to select one of the two or more bridges to be associated with the at least one vital-sign monitor patch.

14. The system of claim 13, wherein the selection is based on signal strengths of respective wireless communication links between the at least one vital-sign monitor patch and the two or more bridges.

15. The system of claim 13, wherein the selection is based on numbers of respective existing associations of the two or more bridges.

16. The system of claim 13, wherein the surveillance server is further configured to prevent one or more unselected bridges among the two or more bridges from communicating with the at least one vital-sign monitor patch.

17. The system of claim 13, wherein the surveillance server is further configured to detect loss of communication connection between the surveillance server and the selected bridge.

18. The system of claim 17, wherein if the loss of communication connection between the surveillance server and the selected bridge is detected, the surveillance server is further configured to select another bridge among the two or more bridges to be associated with the at least one vital-sign monitor patch.

19. The system of claim 13, wherein the surveillance server is further configured to detect loss of wireless communication link between the selected bridge and the at least one vital-sign monitor patch.

20. The system of claim 19, wherein if the loss of wireless communication link between the selected bridge and the at least one vital-sign monitor patch is detected, the surveillance server is further configured to select another bridge among the two or more bridges to be associated with the at least one vital-sign monitor patch.
